(19) United States
(12) Patent Application Publication
(10) Pub. No.: US 2012/0208718 A1
(43) Pub. Date: Aug. 16, 2012

(54) SCHIZOPHRENIA TREATMENT RESPONSE BIOMARKERS

(75) Inventors: Judith Morse Gault, Aurora, CO (US); Ann Olincy, Morrison, CO (US); Robert House, Centennial, CO (US); Bifeng Gao, Highlands Ranch, CO (US)

(73) Assignee: THE REGENTSN OF THE UNIVERSITY OF COLORADO, A BODY CORPORATE, Denver, CO (US)

(21) Appl. No.: 13/499,672

(22) PCT Filed: Oct. 1, 2010
(86) PCT No.: PCT/US10/51194

§ 371 Date: Apr. 18, 2012

(30) Foreign Application Priority Data
Oct. 1, 2009 (US) 61247871

(51) Int. Cl. C40B 30/04; C12Q 1/68; C40B 40/06

(52) U.S. Cl. 506/9; 506/16; 435/6.12

(57) ABSTRACT

The present invention provides biomarker of antipsychotic treatment response in patients with schizophrenia and other disorders involving DRD2 and methods for using the same.

### CROSS-REFERENCE TO RELATED APPLICATIONS

This application claims the priority benefit of U.S. Provisional Application No. 61/247,871, filed Oct. 1, 2009, which is incorporated herein by reference in its entirety.

### STATEMENT REGARDING FEDERALLY FUNDED RESEARCH

This invention was made with government support under Grant No. 1RC1MH088735 awarded by the National Institutes of Health. The government has certain rights in the invention.

### FIELD OF THE INVENTION

The present invention relates to biomarker of antipsychotic treatment response in patients with schizophrenia and methods for using the same.

### BACKGROUND OF THE INVENTION

For diseases like diabetes and hypothyroidism, a simple blood test is routinely used to achieve optimal doses of medication. For diseases like schizophrenia, doses of antipsychotics are inadequately determined by subjective means (e.g., questioning and observing a patient) and trial and error. Each error in medication dose subjects the patient to a likely relapse. Relapse is significant and may cost the patient life, family support, job and home. The optimal dose of any antipsychotic is not truly known. It had been largely accepted that new atypical antipsychotics had superior efficacy. Some studies have shown that patients had a similar response to typical and atypical antipsychotics. Without being bound by any theory, these findings are consistent with the belief that primary antipsychotic action of conventional drugs results from occupancy at dopamine D2 receptor (DRD2). It is also generally believed that the discrepancy between findings of these studies and previous findings relates to uncertainty about the best dose of any particular antipsychotic drug. Discovered over 50 years ago, it is clear that antipsychotics acting as antagonists at DRD2 are effective. The gap in scientific knowledge is in understanding the complete pathway and in knowing which key molecules can assist in optimizing treatment of schizophrenia.

It is estimated that twenty-six percent of adults suffer from mental disorders. Neuropsychiatric conditions including schizophrenia are the primary contributors to disease burden in the United States for individuals aged 15 to 44 years and they contribute about 3 times more to the burden of disease than all cancers combined according to the World Health Organization. Disease burden is an estimate of disability-free life that is lost due to disease and ineffective treatment of disease. Relapse is a primary contributor to schizophrenia disease burden and 61% of patients with schizophrenia have a relapsing and remitting course. Approximately 60% of patients with schizophrenia improve with medication but most remain disabled. Relapse is not entirely due to noncompliance and 27-50% of patients on injectable antipsychotic medication relapse each year into acute psychosis consistent with a need to adjust medications in accordance with a variable disease course. Currently, a patient's dose is titrated to response within a recommended dose range. Serum blood levels can be monitored to take into account factors that change drug metabolism like smoking. However, to date, biological markers of illness or indicators of fluctuation of disease course or markers of response to therapy do not exist.

Therefore, there is a need for developing biological markers for monitoring antipsychotic treatment response in patients with schizophrenia.

### SUMMARY OF THE INVENTION

Schizophrenia is believed to be caused in part by excessive stimulation of pyramidal neurons by dopamine and antipsychotics are DRD2 antagonists. Antipsychotics have similar clinical efficacy and the therapeutic window is achieved by 50-80% DRD2 occupancy. The present inventors have discovered novel isoforms of the DISC1 gene that play a role in the DRD2 pathway. The present inventors have also discovered that decreased expression levels of these DISC1 isoforms are associated with improvement in psychosis in patients with schizophrenia.

The present inventors have identified antipsychotic treatment-response biomarkers in biological samples (e.g., peripheral blood leukocytes) of subjects. These biomarkers were expressed at high levels during acute psychosis then decreased in response to treatment. The treatment-response biomarkers of the present invention are isoforms of the disrupted in schizophrenia 1 (DISC1) gene, named DISC1-biomarkers as used herein. It has been shown that transcription of the DISC1-biomarker changes in response to effective treatment in patients with schizophrenia. In some embodiments of the invention, analysis of DISC1-biomarker exons and/or genotype-phenotype allows one to determine the effectiveness of a pharmacotherapy treatment of schizophrenia.

Some aspects of the invention provide methods for predicting effectiveness of a pharmacotherapy treatment in a schizophrenia patient by determining the expression level of one or more of these DISC1 isoforms or biomarkers. Such expression level can be determined, for example, by measuring the patient's genetic (e.g., transcriptional) response to medications. These biomarkers of treatment-response can be used as an indicator of treatment success or ineffectiveness and can lead to the ability to further optimize the dose of medication in patients with schizophrenia to prevent relapse.

### BRIEF DESCRIPTION OF THE DRAWINGS

FIG. 1 is a schematic illustration of the Wnt/β Catenin Signaling Ingenuity pathway showing 18 of the 45 genes that were differentially expressed between 6 paired samples using gene expression microarray analysis.

FIG. 2 is a schematic illustration of DISC1 isoforms with DISC1-biomarker. Unique regions are all of area “1” and open box region in area “3” and the GSK3β domain is the large shaded box in area “2”.

FIG. 3 is a gel showing the qRT-PCR DISC1-biomarker 216 by PCR product in various cells.

FIG. 4 is a schematic illustration of select transcripts of the DISC1 and TSNAX genes including isoforms, TSNAX-DISC1, TSNAX-DISC1 variant kaje, DISC1 variant q, AK025293 and AK023443, with the region beyond exon 3.

FIG. 5 is a sequence list of TSNAX-DISC1 variant kaje where the double slashes indicate an exon border and the lower case lettering is the 3′UT region.

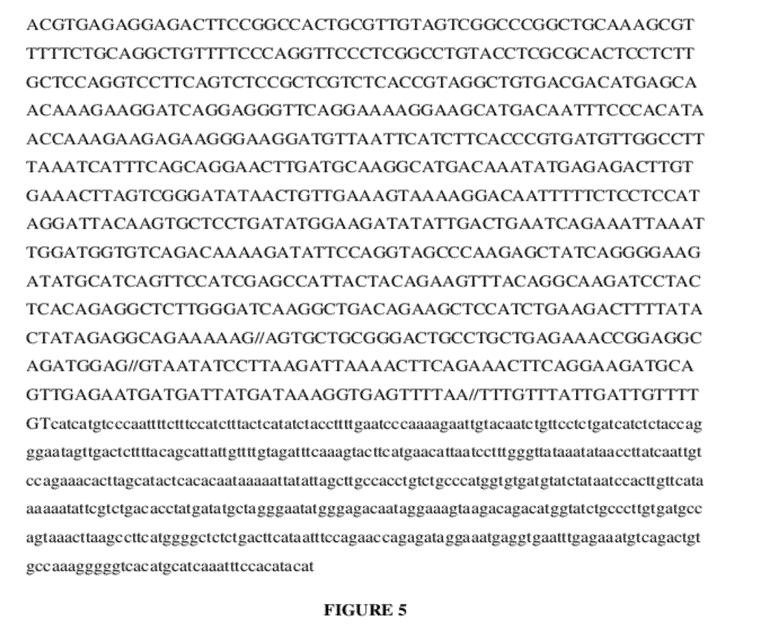

### DETAILED DESCRIPTION OF THE INVENTION

Many dopamine receptors including DRD2 are expressed in peripheral blood lymphocytes. In studies, it has been shown that increased DRD2 mRNA levels in peripheral blood lymphocytes may be used as a diagnostic biomarker for 13 medication-naive male and female patients with schizophrenia but not in 30 patients with variable treatment histories indicating that DRD2 receptor levels on lymphocytes are altered by antipsychotic treatment. This evidence shows that some treatment-response biomarkers are related to the DRD2 pathway and that some patients have variable levels of DRD2. It is believed that a treatment-response biomarker downstream of DRD2 should also measure stimulation of DRD2. The present inventors have discovered that a treatment-response biomarker in lymphocytes that is part of a regulatory feedback loop downstream of DRD2 (e.g., DISC1-biomarkers) are more reliable indicators of treatment success.

Discovery that a large Scottish family with a balanced translocation breakpoint in the Disrupted in Schizophrenia 1 (DISC1) gene that cosegregates with all 7 family members with schizophrenia and 10 other members with major depression, is a good indication that DISC1 is involved in schizophrenia (LOD score 7.1). When identifying causative genes in Mendelian diseases, the focus is typically on the identification of mutations that clearly disrupt the gene in more than one family and co-segregate with disease. There appears to be clinical heterogeneity and reduced clinical penetrance of DISC1 in the two families described with 2 different mutations (the second 4 by deletion occurs in the coding region of DISC1). Several DISC1 haplotypes are associated with schizophrenia as well. DISC1 haplotypes with the small nucleotide polymorphism (SNP) rs3738401 are associated with schizophrenia. SNP rs3738401 results in a change at amino acid number 264 (arginine to glutamine) in exon 2 near the GSK3β binding region. SNPS in exon 2 have been shown to be common to all 5 isoforms of DISC1 including the DISC1-biomarker isoform.

Multiple proteins have been identified that interact with DISC1 during neurodevelopment specifically regulating Grb2 into axons as a cargo receptor and neurotrophin-induced axon elongation. As in the DRD2 pathway, transcripts from genes in the DISC1 pathway are expressed in peripheral blood lymphocytes including DISC1 isoforms L, isoforms ending with exon 11, 10, 9a, 9b, 9, 6, 4, 3a and exon 3 as well as genes shown to interact with DISC1 including GRB2, KIF5A, FEZ1, TRAF3IP1, ATF4, NDEL1, PAFAH1B1, PDE4B, KLC1, NDE1, PCM1 and BBS4. Some of the genes in the pathway including ARRB2, PDPK1, AKT1, NFATC, CTNNB1, ADCY, PRKACA, PPP3CA, CSNK1A1, CDK5, CSNK2 and PPP1CA with correlated gene expression are differentially expressed and associated with effective treatment similar to DISC1 isoforms (i.e., DISC1 biomarkers).

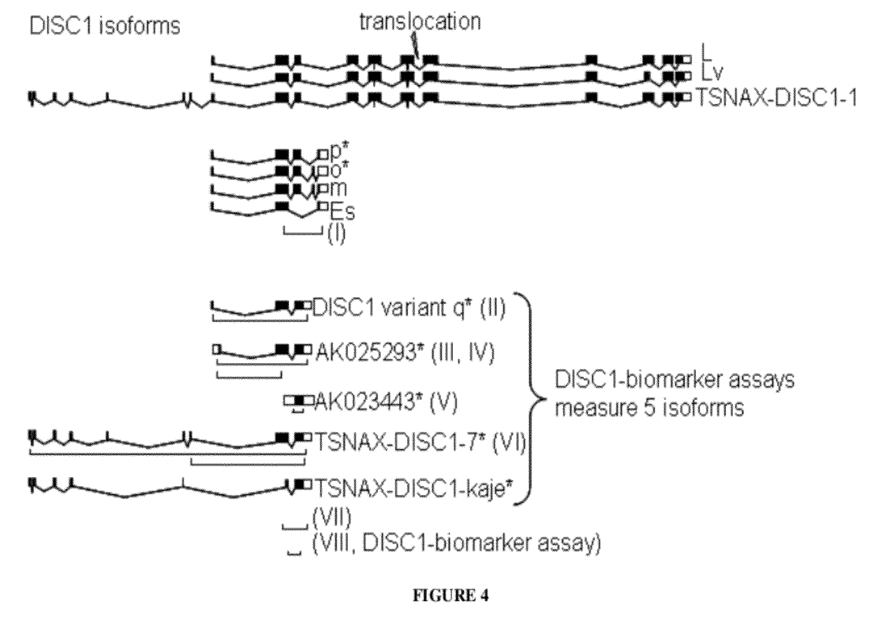

The present inventors have identified antipsychotic treatment-response biomarkers that are expressed at high levels during acute psychosis then decrease in response to pharmacotherapy treatment. The treatment-response biomarkers of the present invention include a previously unstudied isoform of the disrupted in schizophrenia 1 (DISC1) gene referred herein as TSNAX-DISC1 variant kaje (FIGS. 4 & 5), as well as other DISC1 isoforms including variant q, AK025293, AK023443 and TSNAX-DISC1. As discussed above, there is genetic evidence supporting DISC1 involvement in schizophrenia susceptibility. It has been shown that the N terminal of the largest and most studied isoform of DISC1 physically interacts with glycogen synthase kinase-3 beta (GSK3β). The DISC1-biomarker transcripts of the invention include the N-terminal that binds GSK3β. GSK3β is also regulated through the dopamine D2 receptor (DRD2) pathway. Antipsychotics are antagonists at DRD2. DRD2, GSK3β and DISC1 pathways are present in peripheral blood lymphocytes. The present inventors have discovered that some DISC1 isoforms can be used as biomarkers for determining effectiveness of or determining the response to a pharmacotherapy treatment. The present inventors have also discovered additional biomarkers of psychosis from the DISC1/Dopamine/Wnt/Neuregulin pathways that converge on GSK3β.

It is believed that some biological samples (e.g., lymphocytes) from patients with schizophrenia have higher levels of the DISC1-biomarker during acute psychosis when compared to levels after treatment with antipsychotics. In addition, the present inventors have observed that the level of DISC1 isoforms of the invention were lower in patients responding to a pharmacotherapy treatment compared to the DISC1 isoform level in schizophrenia patients during acute psychosis. Moreover, in some instances DISC1-biomaker is expressed at high levels in T cells.

Some aspects of the invention provide biomarker of active psychosis among persons with schizophrenia. Such knowledge can be used to determine pharmacotherapeutic mechanisms, schizophrenia disease mechanisms and the development of a marker of acute psychosis, thereby allowing one to determine suitable dosing. This treatment-response biomarker, the DISC1 gene (Disrupted in Schizophrenia 1), has been associated with the development of severe mental disorders, including bipolar disorder and schizophrenia. Thus, biomarkers of the invention can also be used to determine the effectiveness of or responsiveness to a pharmacotherapy treatment for these other mental disorders.

The present inventors have observed at least 40% reduction in the expression level of a previously uncharacterized DISC1 isoforms after treatment of acute psychosis in patients with schizophrenia. Accordingly, some methods of the invention include determining differences in gene expression between paired lymphocyte samples from the same individual with schizophrenia, while psychotic and while in remission or in pharmacotherapy treatment. It was found that the expression of DISC1 isoforms of the invention was significantly elevated in patients during acute psychosis. For example, the present inventors have discovered increased levels of the DISC1 isoform transcripts during the state of acute psychosis in peripheral lymphocytes of a schizophrenia patient. In some instances, the first blood was drawn during an acute psychotic episode, and the subsequent blood sample(s) were drawn after the patient had stabilized on medication. In other instances, levels of DISC1-biomarker expression were characterized in a healthy normal population and compared to levels in patients during active psychosis and after treatment using qRT-PCR.

Some aspects of the invention provide methods for determining response to or the effectiveness of a pharmacotherapy treatment in a subject undergoing an antipsychotic pharmacotherapy that target DRD2 or a subject suffering from a clinical condition associated with an abnormality in DRD2/GSK3β/DISC1 pathway. Accordingly, in some embodiments, methods of the invention are suitable for determining effectiveness of an/or responsiveness to pharmacotherapy treatment in subjects suffering from schizophrenia, bipolar disorder and major depressive disorder with symptoms of psychosis. It should be appreciated that in generally any clinical conditions associated with an abnormality in DRD2/GSK3β/DISC1 pathway or clinical conditions that can be treated with a DRD2 antagonist are suitable for methods of the invention.

Typically, methods of the invention include determining the expression level of a biomarker associated with a pharmacotherapy treatment response in such patients. Generally, the expression level of biomarker is measured from the patient's own biological sample, such as blood, serum, a tissue sample, lymphocytes, T-cells, etc. Often the biomarker comprises at least one DISC1 isoform disclosed herein. Methods of the invention include comparing the expression level of the biomarker detected in the biological sample to a level of expression of the biomarker in a control to determine the patient's response to or the effectiveness of the pharmacotherapy treatment. In determining the effectiveness of a pharmacotherapy treatment, typically the level of expression of the biomarker in the control comprises the level of expression of the biomarker in the patient during an acute psychosis. However, it should be appreciated that the comparison can also be made to the expression level in a normal control, e.g. expression level in those not affected with an acute psychosis. As stated above, typically a lower level of expression of the biomarker compared to the level of expression of the biomarker in during an acute psychosis period of the patient is indicative of the effectiveness of pharmacotherapy.

In some embodiments, the step for determining the expression level of the biomarker comprises analyzing a plurality of DISC1 isoforms. Often in such embodiments, the expression level of at least four, and more often at least five, DISC1 isoforms is analyzed. In some particular embodiments, the biomarker comprises various DISC1 isoforms such as, but not limited to, DISC1 variant q, AK025293, AK023443, TSNAX-DISC1, TSNAX-DISC1 variant kaje, and a combination thereof. There are a variety of methods for determining the expression level of DISC1 isoforms including, but not limited to, determining the level of mRNA, protein, or gene expression associated with the DISC1 isoforms. In one particular embodiment, the expression level or translation level of mRNA of DISC1 isoform(s) is determined. In other embodiments, biomarkers of the invention comprise at least one DISC1 isoform comprising a variant in exon 3 of DISC1 gene.

Any biological sample that includes DISC1 isoforms can be used in methods of the invention. Typically, the biological sample comprises peripheral blood mononuclear cells of the patient. Often the biological sample comprises a peripheral blood lymphocyte of the patient.

It has been found by the present inventors that a lower expression level of DISC1 isoforms of the invention compared to the level of same DISC1 isoforms in a control subject who exhibits acute psychosis is an indication that the patient is responding positively to the pharmacotherapy treatment or that the pharmacotherapy treatment is effective in reducing the clinical symptoms associated with an abnormality in DRD2/GSK3β/DISC1 pathway. Alternatively, if the expression level of DISC1 isoforms in a subject is similar to the DISC1 isoform expression level of other patients or normal control groups not exhibiting acute psychosis, then it is also an indication that the subject is responding positively to the pharmacotherapy treatment. Or if the DISC1 isoform expression level of the subject is significantly decreased compared to the subject's own DISC1 isoform expression level during the subject's acute psychosis period, then it is also an indication that the subject is responding positively to the pharmacotherapy treatment.

In general, methods of the invention allow one skilled in the art to determine whether a patient is responding positively to a pharmacotherapy treatment in a more objective manner rather than having to rely on a subject test and/or observation. It should be appreciated that levels of DISC1 isoform expression can be determined by any one of the variety of methods known to one skilled in the art, such as by determining the level of any portion of the protein, mRNA, gene expression, or ligand that can identify or correlate with the level of DISC1 isoforms of the invention.

Methods of the invention include detecting or determining whether there is any significant difference in the expression level of DISC1 isoforms in the tested subject compared to the expression level of DISC1 isoforms in a control as discussed above. According to the invention, a “baseline” or “control” can include a normal or negative control and/or a disease or positive control, against which a test level of DISC1 isoform expressions can be compared. Therefore, it can be determined, based on the control or baseline expression level of DISC1 isoforms, whether a pharmacotherapy treatment to be evaluated for positive response has a measurable difference or substantially no difference in the DISC1 isoform expression level, as compared to the baseline (or control) level. In one aspect, the baseline control is an indicative of the expression level of DISC1 isoforms as expected in a normal (e.g., healthy, negative control, or psychotic) patient. Therefore, the term “negative control” used in reference to a baseline or control expression level of DISC1 isoforms typically refers to, but not limited to, a baseline expression level of DISC1 isoforms from a population of individuals which is believed to be normal (i.e., not suffering acute psychosis).

In some embodiments of the invention, a patient's test sample is compared to the control DISC1 isoform expression level that has previously been established from the patient itself during acute psychosis period or from population of patients during acute psychosis period. Such a baseline expression level, also referred to herein as a “positive control”, refers to an expression level of DISC1 isoforms established from one or often a population of patients at the time of acute psychosis.

In some embodiments, the control or baseline expression level of DISC1 isoforms is obtained from “matched individuals”. The phrase “matched individuals” refers to a matching of the control individuals on the basis of one or more characteristics, such as gender, age, race, or any relevant biological or sociological factor that may affect the baseline of the control individuals and the patient (e.g., preexisting conditions, consumption of particular substances, levels of other biological or physiological factors). The number of matched individuals from whom control samples must be obtained to establish a suitable control level (e.g., a population) can be determined by those of skill in the art, but should be statistically appropriate to establish a suitable baseline for comparison with the patient to be evaluated (i.e., the test patient). The values obtained from the control samples are statistically processed using any suitable method of statistical analysis to establish a suitable baseline level using methods standard in the art for establishing such values. It will be appreciated by those of skill in the art that a baseline need not be established for each assay as the assay is performed but rather, a baseline can be established by referring to a form of stored information regarding a previously determined control expression level of DISC1 isoforms. Such a form of stored information can include, for example, but is not limited to, a reference chart, listing or electronic file of population or individual data regarding “normal” (negative control) or positive DISC1 isoform expression level; a medical chart for the patient recording data from previous evaluations; or any other source of data regarding control DISC1 isoform expression level that is useful for the patient to be diagnosed or evaluated.

Expression of the transcripts and/or proteins encoded by the DISC1 isoform genes can be measured by any of a variety of known methods in the art. In general, the nucleic acid sequence of a nucleic acid molecule (e.g., DNA or RNA) in a patient sample can be detected by any suitable method or technique of measuring or detecting gene sequence or expression. Such methods include, but are not limited to, polymerase chain reaction (PCR), reverse transcriptase-PCR (RT-PCR), in situ PCR, quantitative PCR (q-PCR), in situ hybridization, Southern blot, Northern blot, sequence analysis, microarray analysis, detection of a reporter gene, or other DNA/RNA hybridization platforms. For RNA expression, typical methods include, but are not limited to, extraction of cellular mRNA and Northern blotting using labeled probes that hybridize to transcripts encoding all or part of DISC1 isoform; amplification of mRNA expressed from DISC1 isoform using gene-specific primers, polymerase chain reaction (PCR), quantitative PCR (q-PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene. The term “quantifying” or “quantitating” when used in the context of quantifying transcription levels of DISC1 isoforms can refer to absolute or to relative quantification. Absolute quantification can be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals to quantify the changes in hybridization intensity and, by implication, transcription level.

Methods to measure protein expression levels of DISC1 isoforms include, but are not limited to, Western blot, immunoblot, enzyme-linked immunosorbant assay (ELISA), radioimmunoas say (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), flow cytometry, and assays based on a property of DISC1 isoforms including but not limited to ligand binding, or interaction with other protein partners.

Nucleic acid arrays can also be used for detecting the expression of DISC1 isoforms. The production and application of arrays in gene expression monitoring have been disclosed previously in, for example, PCT Publication Nos. WO 97/10365, WO 92/10588, WO 95/35505, U.S. Pat. Nos. 6,040,138 and 5,445,934, Hacia et al. (1996) Nature Genetics 14:441-447, Lockhart et al. (1996) Nature Biotechnol. 14:1675-1680, and De Risi et al. (1996) Nature Genetics 14:457-460, all of which are incorporated herein by reference in their entirety. In general, in an array, an oligonucleotide, a cDNA, or genomic DNA, that is a portion of DISC1 isoform, occupies a known location on a substrate. A nucleic acid target sample is hybridized with an array of such oligonucleotides and then the amount of target nucleic acids hybridized to each probe in the array is quantified. One exemplary quantifying method is to use confocal microscope and fluorescent labels. The Affymetrix GeneChip™ Array system (Affymetrix, Santa Clara, Calif.) and the Atlas™ Human cDNA Expression Array system are particularly suitable for quantifying the hybridization; however, it will be apparent to those of skill in the art that any similar systems or other effectively equivalent detection methods can also be used. One can use the knowledge of DISC1 isoforms disclosed herein to design arrays of polynucleotides, cDNAs or genomic DNAs for screening methods described herein. Such novel pluralities of polynucleotides are contemplated to be a part of the invention.

In general, typical clinical samples include, but are not limited to, blood or blood cells such as white blood cells (e.g., granulocytes and monocytes), buccal swabs, tissues, urine, saliva, etc.

The expression level of DISC1 isoforms can also be determined by conjugation or ligand-binding interaction using a DISC1 isoform ligand and/or DISC1 isoform antibody that is detectably marked. Detectable markers suitable for use in the invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, infrared dyes and the like), radiolabels (e.g., ³H, ¹²⁵I, ³⁵S, ¹⁴C, C or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

Another embodiment of the invention relates to a plurality of antibodies, or antigen binding fragments thereof, for the detection of DISC1 isoforms in samples of the subject. The plurality of antibodies, or antigen binding fragments thereof, consists of antibodies, or antigen binding fragments thereof, that selectively bind to DISC1 isoforms. In addition, the plurality of antibodies, or antigen binding fragments thereof, comprises antibodies, or antigen binding fragments thereof, that selectively bind to DISC1 isoforms or portions thereof.

The phrase “selectively binds to” refers to the ability of an antibody, antigen binding fragment or binding partner (antigen binding peptide) to selectively bind to DISC1 isoforms. Often the phrase “selectively binds” refers to the specific binding of DISC1 isoforms (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

Limited digestion of an immunoglobulin with a protease may produce two fragments. An antigen binding fragment is referred to as an Fab, an Fab′, or an F(ab′)₂ fragment. A fragment lacking the ability to bind to antigen is referred to as an Fc fragment. An Fab fragment comprises one arm of an immunoglobulin molecule containing a L chain (V_{L}+C_{L} domains) paired with the V_{H} region and a portion of the C_{H} region (CH1 domain). An Fab′ fragment corresponds to an Fab fragment with part of the hinge region attached to the CH1 domain. An F(ab′)₂ fragment corresponds to two Fab′ fragments that are normally covalently linked to each other through a di-sulfide bond, typically in the hinge regions.

Isolated antibodies of the invention can include serum containing such antibodies, or antibodies that have been purified to varying degrees. Whole antibodies of the invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), can also be employed in the invention.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies can be produced according to the methodology of Kohler and Milstein (Nature, 1975, 256, 495-497). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

Methods of the invention can include a step of comparing the results of detecting or determining the DISC1 isoform expression level in the subject with the DISC1 isoform expression level in a control (baseline, normal control or patient during acute psychosis period) in order to determine whether there is any difference in the DISC1 isoform expression level in the subject as compared to the control. As discussed herein, the DISC1 isoform expression level can be compared to a “normal” or “negative” control (i.e., a subject that is not suffering acute psychosis) or a “positive” control (i.e., a subject during an acute psychosis period). Therefore, one can determine whether the DISC1 isoform expression level from the test subject is statistically substantially similar to the DISC1 isoform expression level of subjects during an acute psychosis period or whether the DISC1 isoform expression level in the test subject is statistically more similar to the negative or normal control.

In many instances, the DISC1 isoform expression level is substantially similar to a given DISC1 isoform expression level established for a group (e.g., a group of patients or the patient itself during an acute psychosis period, or normal or “negative” control group) if the DISC1 isoform expression level determined or detected is similar enough to the expected result so as to be statistically significant (e.g., with at least a 95% confidence level, or p<0.05, typically with a confidence level of p<0.01, and often with a confidence level of p<0.005, and more often with a confidence level of p<0.001). Software programs are available in the art that are capable of analyzing whether the difference between the DISC1 isoform expression level from the test subject and a control is significant or not significant. In addition, statistical analysis methods are well known in the art.

The DISC1 isoform expression level in a patient can be used by the patient or physician for decision-making regarding the usefulness of that particular pharmacotherapy treatment in general. The DISC1 isoform expression level can be used to estimate the effectiveness of the pharmacotherapy treatment in that particular patient. The DISC1 isoform expression level can also be used to determine patient's suitable dosage to a particular antipsychotic drug.

Yet other aspects of the invention provide microarrays comprising a plurality of oligonucleotides that are capable of detecting expression level of at least two DISC1 isoforms selected from the group consisting of DISC1 variant q, AK025293, AK023443, and TSNAX-DISC1. In some embodiments, such microarrays are capable of detecting the expression of at least three and often all four DISC1 isoforms provided. As used herein, the term “microarray” refers to any ordered sets of oligonucleotides of known sequence. Each individual feature goes on the array at precisely defined location on the substrate. Exemplary microarray include a 2D array, typically on a glass, filter, micro-wells, or silicon wafer, upon which oligonucleotides are attached or synthesized in a predetermined spatial order allowing them to be made available as probes in a high-throughput, parallel manner.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

The patients were invited to participate in the study within 1-3 days of being hospitalized for acute psychosis and after being diagnosed with schizophrenia. The patients had already started antipsychotic treatment before being enrolled. A Structured Clinical Interview for DSM-IV Axis (SCID-I/P) was administered to confirm that the patients met the criteria for schizophrenia. A brief psychiatric rating scale (BPRS) was administered to assess symptoms and blood was collected in 5 sodium heparin tubes (for lymphocytes), 1 Tempus tube (for total blood RNA), and 2 EDTA tubes (for DNA and complete blood count). The second of the paired blood draws occurred once the patient had recovered and was ready to leave the hospital. The average time between paired sample collection was 5.8 days. Again the BPRS was performed by the same person (PRA) who collected blood in 1 EDTA tube, 1 Tempus tube and 5 sodium heparin tubes. Both blood draws occurred around the same time to avoid time of day (circadian rhythm) influences on the data. Improvement in the patient between the collection times was usually obvious although the patients do not completely recover and remain impaired. BPRS analysis of 30 paired samples collected from consecutively enrolled patients over 16 months showed significant improvement in positive symptoms (p=0.016) and in the total BPRS score (p=0.002). In fact, 29 of the 30 the patients showed noticeable improvement in their psychotic symptoms by the second blood draw. The symptoms of one patient got worse by the second blood draw so a third blood draw was obtained.

Patients whose psychosis did not improve between blood draws are not included in the additional studies unless a third sample was collected when the patient improved. The 49 patients that were enrolled over 16 months consisted of 30 paired samples that were not available for further study. Of the 30 paired samples collected, patient information of 12 paired samples are listed in Table 1. These paired samples were used to identify DISC1 isoforms as treatment-response biomarkers. BPRS analysis of the 12 paired samples showed a significant improvement in positive symptoms (p=0.037) and in the total BPRS score (p=0.020) (Table 1, columns 3 and 4) indicating that the DISC1 isoforms are good biomarkers for determining improvement in state of psychosis. The other 18 samples were not suitable because CD4+ lymphocytes were isolated for other studies (14 samples), the RNA quality was compromised in one of the paired samples (2 of the 30 paired samples) or the preparations of lymphocytes were contaminated with red blood cells (2 of the 30 paired samples).

TABLE 1
Patient Information

| | Days | | | | Medication | |
|---|---|---|---|---|---|---|
| | between | BPRS 1^{st} | BPRS 2^{nd} | Current | Just before/during | |
| Age | Samples | (+/total) | (+/total) | Smoker | hospital stay | Other |
| 44 | 10 | 21/53 | 7/33 | yes | Risperidone, Depakote, | Drugs not detected, |
| | | | | | Olanzapine | Medication |
| | | | | | | noncompliant |
| 37* | 3 | 24/56 | 15/46 | yes | Aripiprazole | Marijuana+, Homeless |
| 59* | 2 | 16/50 | 6/36 | no | Haloperidol, | Drugs not detected, |
| | | | | | Olanzapine | Medication compliant |
| | | | | | | Homeless |
| 54* | 2 | 17/61 | 8/22 | yes | Haloperidol, | Marijuana+, |
| | | | | | Risperidone | Medication |
| | | | | | | noncompliant |
| 45* | 7 | 9/28 | 10/28 | yes | Quetiapine, Depakote, | Cocaine + |
| | | | | | Olanzapine | Medication compliant |
| | | | | | | Homeless |
| 53* | 6 | 20/54 | 6/25 | yes | Aripiprazole, Depakote | Drugs not detected |
| | | | | | Quetiapine Paxil | |
| 39* | 15 | 18/60 | 14/40 | yes | Aripiprazole, | Drugs not detected, |
| | | | | | Olanzapine | Medication |
| | | | | | | noncompliant Homeless |
| 38* | 3 | 18/60 | 13/50 | yes | Fluphenazine, | Marijuana + |
| | | | | | Olanzapine, Lithium, | Medication |
| | | | | | Albuterol, Azmacort | noncompliant Homeless |
| 59* | 2 | 6/34 | 10/37 | no | Quetiapine, Celexa, | Medication compliant |
| | | | | | Nexium, Tegretol, | |
| | | | | | Sinemet | |
| 45* | 14 | 14/23 | 12/53 | yes | Risperidone, Depakote, | Drugs not detected, |
| | | | | | Trazodone | Medication compliant, |
| | | | | | | Homeless |
| 49* | 2 | 15/39 | 16/34 | yes | Risperidone, | Drugs not detected, |
| | | | | | Haloperidol, Depakote, | Medication |
| | | | | | Inderal, Quetiapine | noncompliant Homeless |
| 58* | 3 | 6/40 | 6/36 | pipe | Aripiprazole, | Drugs not detected |
| | | | | | Quetiapine, Olanzapine, | Possibly noncompliant |
| | | | | | Klonopin, Clonazepam | |

Bold = Gene Chip Samples;
*qRT-PCR samples

DISC1-biomarker levels were determined in all samples (paired and unpaired) collected during acute psychosis for the genetic analysis. The DISC1-biomarker data from samples collected during acute psychosis that were generated was used for further study. The data generated on 11 paired samples were also used. In addition, there were 16 unpaired samples available and additional 35 unpaired samples were used. Overall, DISC1-biomarker data on 92 lymphocyte samples collected during acute psychosis were generated.

Participants were assigned a research number to maintain confidentiality and pertinent information was stored in a computer database. Information collected included age of first symptom, age when diagnosed, where the patient is living and family history of mental illness, the patient's medical history, history of psychosis, medication history, smoking history, alcohol history, any recent cold medicine use, drug use (urine screen upon hospitalization) and caffeine intake was obtained from both the patient and from the patient's medical records.

Blood samples (16 ml) were collected in Becton-Dickinson Vacutainer® sodium heparin tubes (BD, Franklin Lakes, N.J.) around 2 PM. Lymphocytes were isolated as directed using Lymphoprep™ (AXIS-SHEILD, Oslo, Norway) and total RNA was extracted from lymphocytes as directed using TRIZOL® Reagent (Invitrogen). Lymphoprep™ isolation may result in 0.4 to 1.4% of contaminating reticulocytes total RNA (primarily globin mRNA). This method of purification was quick, simple and yielded sufficiently pure lymphocytes. RNA quality was assessed on Agilent Bioanalyzer 2100 (Quantum Analytics, Inc. Foster City, Calif.). RNA were not DNase treated. The RNA samples were sufficient for gene chip analysis and qRT-PCR analysis of 22 genes. Follow-up analysis with qRT-PCR on DISC1-biomarker and up to 15 genes was performed.

DNA Isolation from Blood

A simple salting out procedure was used for samples using a Beckman Coulter Allegra 6 centrifuge (Beckman, Palo Alto, Calif.) to extract genomic DNA from approximately 10 ml of blood collected in tubes with EDTA anticoagulation reagent. Typical yields of DNA were 10-30 μg/ml of blood allowing more than 1000 PCR experiments for each sample.

RNA Isolation from Blood

Blood was collected in Tempus Blood RNA Tubes (Applied Biosystems, Foster City, Calif.) and stored at −20° C. Total RNA was extracted using VERSAGENE™ RNA purification kit (Gentra Systems, Minneapolis, Minn.). RNA was used to investigate splicing mutations and to screen for nucleotide changes.

A complete blood count was done for each sample collected from controls and patients. Samples were collected in Becton-Dickinson Vacutainer® EDTA tubes (BD, Franklin Lakes, N.J.) and a complete blood count including a measurement of the white blood cells was performed. An ANOVA was performed to determine if changes in the white blood cell count were associated with changes in DISC1-biomarker levels.

### Differential Expression in 6 Paired Lymphocytes Using Gene Chip Analysis

Six paired samples were further purified using QIAGEN RNeasy mini kit (QIAGEN, Valencia, Calif.) for gene chip analysis. Quality of RNA was assessed using the Agilent Bioanalyzer 2100 (Quantum Analytics, Inc. Foster City, Calif.). RNA (50 ngs) was amplified and labeled as directed by Ovation™ System (NuGEN Technologies Inc., San Carlos, Calif.). The labeled cRNA was hybridized to Human Genome U133 Plus 2.0 Arrays (Affymetrix, Santa Clara, Calif.). Affymetrix data was normalized using the MASS filter. Principal component analysis (PCA) was performed to visualize overall similarity and variability among the paired samples using Partek Genomic Solutions (Partek, St. Charles, Mo.). Pair wise comparison analysis was done using Affymetrix GCOS v1.4 data analysis software to identify genes similarly up or down regulated across the 6 paired samples. Paired T-test analysis was performed using Partek Genomic Solutions (Partek, St. Charles, Mo.) to determine p-values for the differentially expressed genes.

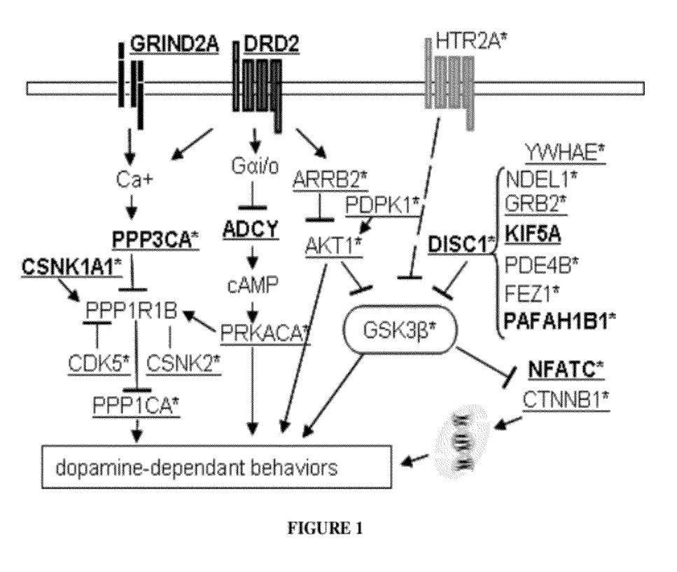

Pair wise analysis indicated there were 468 genes that were similarly differentially expressed during psychosis in at least 3 out of 6 paired samples. Genes similarly up or down regulated in 3 of the 6 paired samples were identified and were further investigated. Pathway analysis of the 468 GCOS-identified genes indicated 11 pathways (p<0.05) using Ingenuity Pathways Analysis (Ingenuity® Systems, Inc., Redwood City Calif.) based on the proportion of the 468 genes differentially expressed within the pathway. Significant pathways included Wnt/catenin pathway (Table 2, FIG. 1) and T cell receptor pathway (Table 3). Twelve genes in the neuregulin pathway were differentially expressed but the pathway did not reach significance and there was decreased DISC1 expression after treatment. Table 2 shows 45 Wnt signaling genes that were differentially expressed in paired samples using gene chip analysis. Of the 45 genes, 27 were unique to the Wnt signaling pathway (columns 1 and 2), 12 were found both in Wnt signaling and PI3K/AKT pathways (column 3, underlined), and 6 were also found in the dopamine pathway (column 4 in italics). Verification of the differential expression in the 44 genes, not including DISC1-biomarker was conducted using gene chip analysis and qRT-PCR analysis.

TABLE 2
List of 45 differentially expressed genes in the GSK3β pathways

| | | Wnt/PI3K/ | Wnt/Dopamine/ |
|---|---|---|---|
| Wnt Signaling | Wnt continued | AKT pathway | PI3K/AKT |
| ACVR1C | MYC | AKT3 | PPP1R3D |
| ACVR2B | PSEN1 | BCL3 | PPP2R5C |
| BTRC | PTPN11 | CTNNB1 | PPP2R5E |
| CD44 | RARA | FOXO3 | PRKAG2 |
| CDH5 | RPS6 | HLA-B | PRKAR1A |
| CSNK1A1 | SMO | ITGA4 | DISC1-Biomarker |
| CSNK2A2 | SOX17 | ITGB1 | |
| ELK1 | SOX5 | MAP3K8 | |
| EREG | SOX6 | MAPK1 | |
| FZD1 | TCF4 | NFKBIA | |
| GNAQ | TCF7L2 | PDPK1 | |
| HBEGF | TGFBR2 | PIK3CB | |
| LEF1 | TLE3 | | |
| LRP1 | | | |

TABLE 3
Gene Expression Analysis in Paired Lymphocytes

| Gene Symbol | Change | Fold-Change | P value* | qRT-PCR* |
|---|---|---|---|---|
| DISC1 | 3/6 | 1.4-1.7↓ | 0.193 | 0.037 |

T Cell Receptor Signaling Pathway

| | | | | |
|---|---|---|---|---|
| CTLA4 | 3/6 | 1.3-1.4↑ | 0.571 | 0.170 |
| PPP3CA | 3/6 | 1.2-1.4↓ | 0.016 | 0.113 |
| NFKBIA | 3/6 | 1.3-2.5↑ | 0.324 | 0.112 |
| JUN | 4/6 | 1.2-2.3↑ | 0.088 | 0.336 |
| FOS | 3/6 | 1.3-1.7↑ | 0.001 | 0.516 |
| DUSP6 | 3/6 | 1.2-1.5↑ | 0.043 | 0.810 |
| LCK | 3/6 | 1.3-1.5↑ | 0.193 | |
| TRAA | 3/6 | 1.2-1.4↓ | 0.416 | |
| RASA1 | 3/6 | 1.3-1.9↑ | 0.732 | |

*two-tailed

Verification of qRT-PCR Assay

Changes in gene expression in 11 paired samples were assessed using qRT-PCR. Results of expression analyses (gene chip and qRT-PCR) are reported in Table 3 for the DISC1-biomarker and for 6 of the 9 genes in the T cell receptor pathway. In Table 3, the column headed by “Change” was the number of samples that were similarly up or down regulated out of 6 tested by gene chip analysis. In Table 3, the column headed by “Fold Change” was calculated by the gene chip analysis. In Table 3, the column headed by “P value” was calculated from the gene chip data using a paired T-test analysis. Paired T-test analysis considered the mean expression change from the combined 6 paired samples. P-values were calculated using a paired T-test analysis performed using Partek Genomic Solutions (Partek, St. Charles, Mo.) software with a MASS filter to normalize the data. In Table 3, the column headed by “qRT-PCR” was based on a paired T-test analysis of qRT-PCR generated data. As can be seen, DISC1-biomarker expression was increased during psychosis (decreased after effective treatment) in 10 of the 11 paired samples using qRT-PCR (40% reduction; p=0.037 using the average of triplicates for each sample and a paired T-test).

RNA was reverse transcribed in triplicate (1 ug) using 0.29 μgs of RNA per tube in a 20 μL reaction. For each sample one reaction without reverse transcription was performed in a 16.8 μL reaction. RNA incubated at 65° C. for 5′, then 4° C. Reverse transcription mix was added and incubated at 25° C. for 10′ then at 42° C. for 1 hour, then 99° C. for 5′. Reaction mix was Superscript™ II (2.5 Units per ul in 1× First-Strand Buffer, 0.01M dithiothreitol (DTT), 8 mM random hexamers (Pharmacia & Upjohn Diagnostics, Kalamazoo, Mich.), and 0.5 U/ml placental RNase inhibitor (Boehringer-Mannheim, Indianapolis, Ind.).

Quantitative PCR was performed using the DISC1 primer set VIII (wild type sequence is an A) designed using PerlPrimer v1.1.14 and normalized with an 18S amplicon. The DISC1 and 18S amplicons had correlation coeffiecients were 0.979 and 0.985 with PCR efficiencies of 89.5% and 97.6% based on analysis of 5 and 9 point standard curves respectively. The cDNA template (2 μL) was qPCRed with the primers at 140 nM final concentrations in 1× SYBR green I dye and fluorescein mix with Thermo-Start DNA polymerase in a total reaction volume of 27 μL (Thermo Fisher Scientific Inc. Waltham, Mass.). Both assays were performed on a BioRad iCycler® (BioRad, Hercules, Calif.) using the following program: 95° C. for 15′; (95° C. for 15″; 60° C. for 1′) for 40 cycles followed by a melt curve. Relative gene expression was determined using both the Livak 2^{-ΔΔC(t)} and Pfaff1 methods with iQ5™ version 2.0 (BioRad, Hercules, Calif.). Significance was tested with a paired T-test. The DISC1-biomarker assay was tested using various control templates including DNA only, DNase treated RNA, poly A isolated RNA, water, RNA without reverse transcriptase. DISC1 isoforms were sought in RNA from PBMCs (FIGS. 1 and 4, Table 4).

RT-PCR analysis was employed to detect the presence of DISC1 isoforms in RNA from PBMCs using primers specific to 4 DISC1 isoforms that included the Affymetrix targeted sequence from probe set 207759_s. See Table 4. Transcripts from 62 DRD2/DISC1 pathway genes (ADCY1, ADCY2, ADCY8, AKT1, AKT1S1, ARRB1, ATF4, CCDC88A, CDK5, CSNK1A1, CSNK2B, CTNNB1, DISC1, DRD1-5, DIXDC1, DVL3, FEZ1, GABRA1-6, GRB2, GRIN2A, GRIN2B, GSK3B, HTR2A, HTRA2, HTR1E, HTR2B, HTR1F, HTRA1, HTRA4, HTR3C, HTR6, HTR7, KIF5A, MAP1A, NDEL1, NFATC1-4, NFACT2IP, PAFAH1B1, PDE4B, PDPK1, PPP1CA, PPP1CB, PPP1CC, PPP1R1B, PPP3CA, PRKACA, TRAF31P1, TSNAX, YWHAE) measured with 165 probe sets on Human Genome U133 Plus 2.0 Arrays (Affymetrix, Santa Clara, Calif.) were correlated using Pearson's on log 2 transformed data generated from 66 CD34+ samples from dataset GDS2118 available through the GEO database.

TABLE 4
Primer sets

| Set | Transcripts | Sequence | Location | bp |
|---|---|---|---|---|
| I | p, o, m, Es | F-TTGGGACACCCTGCTCAGGAAT | DISC1 exon 2 | p = 316 |
| | | R-GTATTCTTCCACGTGGGTCCTCTT | 3′UT | o = 393 |
| II | DISC1q | F1-CAGCCCAGGCGGAGCGGGAGGA | DISC1 exon 1 | |
| | | F2-GGAGCTGGCAGCGGGGCGCATG | DISC1 exon 1 | 1179 |
| | | R1-CCCTGGTAGAGATGATCAGAGGAACA | DISC1 3′UT of | |
| | | | exon 3 | |
| | | R2-GGGACATGATGACAAAACAATC | DISC1 3′UT of | |
| | | | exon 3 | |
| III | AK025293 | F-TCACCCAAGATGCCTCCAGAAGAA(AG) | AK025293 exon | 1271 |
| | | | 1 | |
| | | R1-CCCTGGTAGAGATGATCAGAGGAACA | DISC1 3′UT of | |
| | | | exon 3 | |
| | | R2-GGGACATGATGACAAAACAATC | DISC1 3′UT of | |
| | | | exon 3 | |
| IV | AK025293 | F-TCACCCAAGATGCCTCCAGAAGAA(AG) | AK025293 exon | 481 |
| | | | 1 | |
| | | R-ACCTCTGAGCTGAATCCCAAAGTG(CC) | DISC1 exon 2 | |
| V | AK023443 | F-GAAGAAGTTCAGTTTTGAAACAG | 3′UT of exon 3 | 312 |
| | | R-AACTATTCCCTGGTAGAGATG | | |
| VI | TSNAX-DISC1-7 | F1-TTTCCCAGGTTCCCTCGGCCTGTA | TSNAX exon 1 | |
| | TSNAX-DISC1- | F2-TGAGGAACATGGACGAGATGGGAA | TSNAX exon 7 | 1207 |
| | kaje | | | |
| | | R1-CCCTGGTAGAGATGATCAGAGGAACA | DISC1 3′UT of | |
| | | | exon 3 | |
| | | R2-TCCCTGGTAGAGATGATCAGAGGA | DISC1 3′UT of | |
| | | | exon 3 | |
| VII | TSNAX-DISC1-7 | F-TCACAGTGCCTTTACCTCAAGCTTTAGCT | DISC1 exon 2 | 798 |
| | DISC1q | R-ATGGGCAGACAGGTGGCAAG | DISC1 3′UT of | |
| | AK025293 | | exon | |
| VIII | q, AK025293 | F-GGCAGATGGAG/GTAATATCC | DISC1 exon 2/3 | 216 |
| | AK023443 | R-AACTATTCCCTGGTAGAGGTG | 3′UT of exon 3 | |
| | TSNAX-DISC1-7 | | | |
| IX | 18S | F1-CGGCTACCACATCCAAGGAA | exon 1 | 187 |
| | | R1-GCTGGAATTACCGCGGCT | exon 1 | |

### DISC1-Biomarker Gene Structure

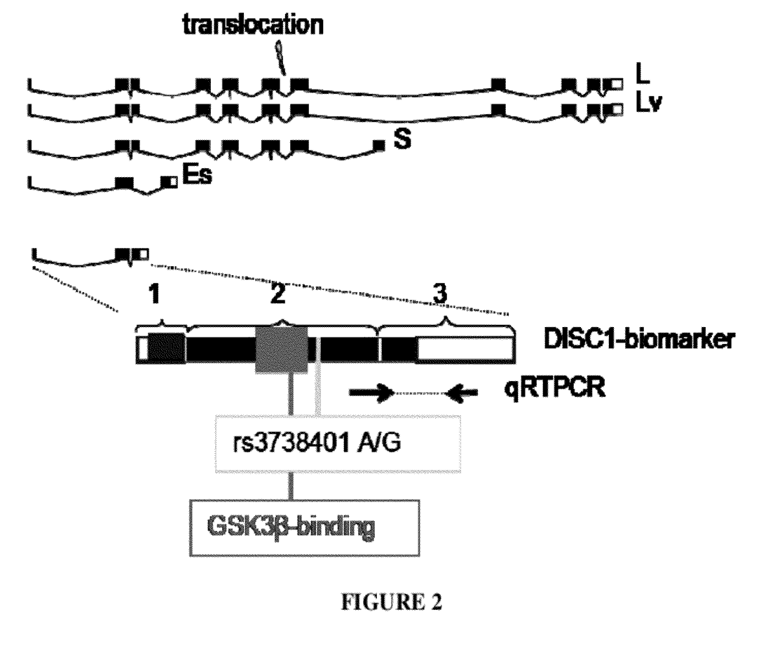

The probe set used on the Affymetrix Gene Chip for DISC1-biomarker targeted a 3 prime untranslated (3′ UT) sequence in exon 3 that was not part of the well characterized isoforms, L, Lv, S and Es (FIG. 2, open region on the right in area “3”). The Affymetrix probe set sequence was part of the 3′UT sequence of cDNA clone AK025293 found in GenBank. In order to verify the gene chip DISC1 results, a qRT-PCR primer set was designed that selectively amplified the DISC1-biomarker (AK025293 sequence). Alignment of clone AK02593 with the other DISC1 isoforms showed that the 5′ UT (145 bp), exon 1 (72 bp) and 3′UT (640 bp) sequences were unique and were not homologous to other DISC1 exons (FIG. 2 shaded region on the left under “1”). Exon 2 and the coding region of exon 3 were identical to exons 2 and 3 found in the other isoforms. Exon 2 includes the GSK3β binding domain (FIG. 2, shown in large shaded block in region “2”) and the SNP rs3738401 in light bar (in region “2”) that results in an amino acid change (rs3738401, nucleotide A791G, amino acid Q264R). Genotype-phenotype correlations with rs3738401 were conducted as described below. The presence of a transcript including exon 2, exon 3 and the 3′UT in lymphocytes was confirmed using RT-PCR and sequence analysis. A forward primer in exon 2 and reverse primer in the unique 3′UT region resulted in an RT-PCR product of the expected size and sequence that matched cDNA AK02593. Sequence analysis of the qRT-PCR product confirmed that it had the expected AK02593 sequence. Experiments confirming that the DISC1-biomarker (AK02593) transcript includes exon 1 sequence are also described below.

### Gene Expression Analysis

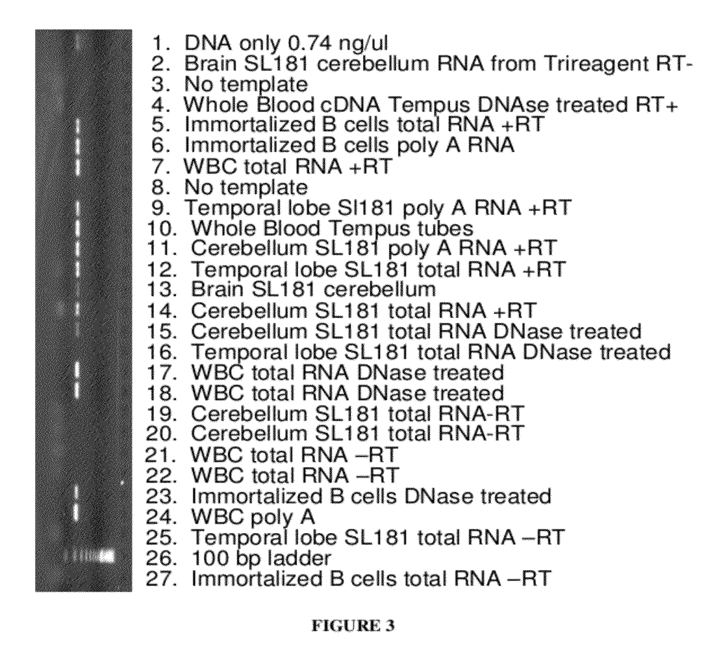

The DISC1-biomarker set can amplify genomic DNA (FIG. 3, lane 1) even though the primer crosses a splice site it included 9 nucleotides of exon 3. All patient samples included a control without reverse transcriptase (RT-) to detect contamination of DNA. None of the controls without reverse transcriptase were positive for the DISC1-biomarker indicating that genomic DNA was not interfering with quantification of the DISC1-biomarker assay. Reverse transcriptase controls need not be included in all assays for every sample and all samples were run in triplicate.

Further investigation into the DISC1-biomarker qRT-PCR assay was performed to verify that the assay detected message in the RNA samples and the amplification was not due to contaminating DNA. Lymphocytes (WBCs) expressed DISC1-biomarker at a higher level then other cells (FIG. 3, lanes 7, 17, 18, 24 with RT-controls in lanes 21 and 22). There was little to no primer dimer formed in the WBC samples. DISC1-biomarker product was present even after DNase treatment (FIG. 3, lanes 17 and 18) and isolation of poly A message (FIG. 3, lane 24) consistent with detection of a transcript. The data and the assay described above used total RNA isolated from WBCs. DNase treatment or poly A isolation were not used because substantially more RNA was required to perform the assay and based on the no reverse transcription controls these steps did not appear to be necessary.

The DISC1-biomarker was expressed in RNA isolated from postmortem temporal lobe (lanes 9, 12, 16 and 25), cerebellum (lanes 11, 14, 15, 19, 20), immortalized B cells (lanes 5, 6, 23 and 27) and whole blood (lanes 4 and 10) (FIG. 3). However, expression in these tissues was lower than what was detected in WBC and these C(t) readings were not on the standard curve. Further analyses were conducted in order to determine expression of DISC1-biomarker in specific lymphocytes and other tissues that are relevant to schizophrenia.

### Results

Forty-nine patients were consecutively enrolled with symptoms of psychosis from December, 2005 to March, 2007. Paired blood samples suitable for this study were collected from 27 of the patients that showed a combined improvement in positive symptoms based on the BPRS using a paired T-Test (P=0.01). High quality RNA was isolated from the PBMCs. However, the first sample collected from one of the patient included some organic solvent so it was only included in the RT-qPCR assay. The paired samples were ranked by improvement in the positive symptoms of the BPRS score and the rater's overall impression of whether the patient improved. In an attempt to reduce heterogeneity, the samples were ranked secondarily by sex (there was only one female patient) and treatment with risperidone. The top 6 paired samples were used for gene chip analysis and best 11 samples for qPCR. There was significant improvement in positive symptoms from the BPRS scores from the 12 patients (P=0.01; Table 1) and a trend towards significant improvement in the patient's total BPRS scores between samples (P=0.08). However, for 5 patients improvement in positive symptoms was not obvious during the BPRS interview even though their psychosis was successfully treated during hospitalization. For 3 of the 5 patients their positive symptoms didn't improve based on the BPRS. The 12 patients were 48 years old on average and paired samples were collected an average of 5.8 days apart. All the patients met DSMIV criteria for schizophrenia. One of the patient was diagnosis with schizophrenia and bipolar not specified with less confidence. Smoking was reported by 10 patients and they were allowed smoking breaks during hospitalization. Marijuana was detected in 3 patients, cocaine was detected in one patient and drug testing was not conducted in 2 patients. One patient reported cocaine and marijuana use (drug testing not available) that was thought to contribute to exacerbation of his psychosis. Six patients were homeless before hospitalization.

The twelve patients whose samples were utilized for transcript quantification were being treated with many different antipsychotics during hospitalization (Table 1). Single antipsychotics were prescribed for 5 patients, 2 antipsychotics were prescribed for 4 patients and 3 patients were treated with 3 antipsychotics during hospitalization. Olanzapine was prescribed for 6 patients, risperidone for 5 patients, aripiprazole for 5 patients and quetiapine for 3 patients. Typical antipsychotics, fluphenazine and haloperidol, were each prescribed for single patients. In addition to antipsychotic medication, 4 patients were treated with the mood stabilizer valproic acid (VPA) and 2 patients were prescribed lithium (Li+) (Table 1). Six patients reported not being compliant with their medication before hospitalization and 5 patients reported being compliant with their medication. It could not be determined if one of the patient was compliant with his antipsychotic medication before hospitalization.

Pair wise analysis using Gene Chip Operating Software (GCOS) of lymphocyte RNA from 6 paired samples from male patients with schizophrenia suggested there were 468 genes that were similarly differentially expressed during psychosis in at least 3 out of 6 paired samples. Only one of the 4 DISC1 probe sets on the Affymetrix U133 Plus 2.0 array was differentially expressed. DISC1 probe set (207759_s_at) was found up regulated during acute psychosis 1.4 to 1.7 fold in samples S18, S22 and S40 but not changed in samples S30, S39 and S29 on microarray analysis (p=0.193). The targeted sequence for probe set 207759_s_at, matched 4 unique cDNA sequences, the TSNAX-DISC1 variant 7 and 54 (GI:257153373 and 238066764 respectively), DISC1 variant q and isoform 46 (GI:257153300 and 238066748 respectively), AK025293 (GI:10437780) and AK023443 (GI:10435379) (FIG. 4). Differential gene expression was verified using RT-qPCR using DISC1 set I. Transcripts with exons 2, and the exon 3-3′UT as measured by DISC1 primer set I with RT-qPCR were increased during psychosis (decreased after effective treatment) in 10 of the 11 paired samples using qRT-PCR (40% reduction; p=0.037 using the average of triplicates for each sample and a paired T-test). DISC1 RT-qPCR product generated with set I was verified using sequence analysis. Primer set I can amplify genomic DNA because the forward primer spans exons 2 and 3. However, patient RNA samples without reverse transcriptase were negative. Separate experiments with DNase-treated RNA and poly A isolated RNA isolated from lymphocytes confirmed that the RT-qPCR assay was detecting transcripts. In addition, primer set VII, amplified the expected 798 by product that included the RT-qPCR amplicon sequence from lymphocyte RNA.

Four known isoforms include the region adjacent and just beyond DISC1 exon 3 (3′UT). The presence of DISC1 variant q (primers IV) and AK025293 (primer sets V and VI) transcripts were confirmed using isoform-specific PCR and sequence analysis from RNA isolated from PBMCs. Transcript AK023443 (primer set VIII) was present in DNAse treated RNA isolated from PBMCs. Sequence analysis of DISC1 variant q and AK025293 transcripts reveal an ORF in frame with the amino acid sequences of exons 2 and 3 in the full-length transcript. However, the 5′ end of isoform AK025293, probe set 217330, was not found at high enough levels to be scored present in the 6 paired PBMCs subjected to gene chip analysis consistent with the AK025293 isoform being a minor PBMC transcript. Isoform TSNAX-DISC1 was not detected by RT-PCR of a 2022 by product in poly A and total RNA isolated from PBMCs. Present calls were assigned to the gene chip probe set in 3′ UT adjacent to exon 13 (206090_s_at) in all 6 paired patient samples consistent with detection of DISC1 isoforms, L and/or Lv. Only paired samples from S30 showed an increase in probe set 206090_s_at transcription detection.

Quantitative analysis using GEO database gene transcription data showed DISC1 isoforms detected by probes that include 3′UT region beyond exons 3 were present in CD34+ hematopoietic stem cells and immature dendritic cells (datasets GDS2431, GDS2750 respectively). Transcripts of GSK3β, AKT, ARRB2, and CTNNB1 were also found. Of the datasets with DISC1 isoform expression, DRD2 transcripts was given a marginal call in 2 of 7 CD34+ datasets and a present call in 3 of 3 immature dendritic cell datasets. Datasets generated with DNAse-treated RNA did not have detectable levels of DISC1 isoforms with the 3′UT beyond exon 3.

Sixty-four significant correlations of R>|0.50| were found between expression levels or 27 genes (70 probe sets) within the DRD2/DISC1 pathway in dataset GDS2118 where all 5 DISC1 and 4 DRD2 probe sets were considered present in all 66 samples of CD34+ cells. The GDS2118 dataset was initially generated and used to investigate hematopoietic malignancies {Pellagatti, 2006 #2461. The 66 samples consist of 11 samples from control subjects and 55 samples from patients. Results presented in Table 5 highlight significant correlations of CSNK2B, PPP1CA, CDK5 with levels of AKT1 and the DISC1-biomarker measured with probe set 207759_s_at. Results presented in Table 6 highlight significant correlations found between Es and AK025293 isoforms of DISC1, 2 DRD2 probe sets.

TABLE 5
DISC1-biomarker isoforms correlations in CD34+ cells

| | | | | | | | | DISC1- |
|---|---|---|---|---|---|---|---|---|
| | DVL3 | CSNK2B* | PPP1CA* | CDK5* | TUBB2A* | SPTBN4* | PGK1* | biomarker* |
| DISC1- | −0.54 | −0.58 | −0.53 | −0.52 | −0.60 | −0.56 | −0.50 | — |
| biomarker* | | | | | | | | |
| AKT1* | 0.62 | −0.53 | 0.81 | 0.60 | 0.55 | 0.59 | 0.63 | −0.41 |

Pearson's R,
*PBMC microarray detection

TABLE 6
DRD2/DISC1 correlations in CD34+ cells

| | | | DISC1 | DISC1 |
|---|---|---|---|---|
| | AKT1* | DRD2 | pomEs* | AK025293 |
| DISC1 pomEs* | −0.60 | 0.58 | 1 | 0.71 |
| DISC1 AK025293 | −0.37 | 0.53 | 0.71 | 1 |
| ADCY1 | −0.53 | 0.63 | 0.8 | 0.59 |
| ADCY2 | −0.39 | 0.62 | 0.64 | 0.48 |
| ARRB2* | 0.62 | −0.26 | −0.63 | −0.35 |
| CSNK2A* | 0.76 | −0.38 | −0.62 | −0.53 |
| CTNNB1* | 0.62 | −0.37 | −0.63 | −0.35 |
| DRD3 | −0.49 | 0.32 | 0.51 | 0.67 |
| DRD5 | −0.5 | 0.5 | 0.65 | 0.81 |
| GABRA1 | −0.59 | 0.58 | 0.83 | 0.66 |
| GABRA4 | −0.51 | 0.57 | 0.78 | 0.58 |
| GNB1* | 0.65 | −0.35 | −0.61 | −0.45 |
| GRB2* | 0.61 | −0.4 | −0.52 | −0.6 |
| GRIND2A | −0.39 | 0.73 | 0.65 | 0.63 |
| GRIPAP1* | 0.59 | −0.53 | −0.63 | −0.50 |
| HTR1B | −0.44 | 0.59 | 0.6 | 0.48 |
| HTR1E* | −0.62 | 0.59 | 0.83 | 0.59 |
| HTR3A | −0.43 | 0.61 | 0.69 | 0.61 |
| HTR3C | −0.46 | 0.71 | 0.75 | 0.67 |
| KIF5A | −0.66 | 0.5 | 0.69 | 0.84 |
| KLC1* | 0.68 | −0.53 | −0.72 | −0.64 |
| MACF1* | 0.19 | −0.62 | −0.55 | −0.44 |
| MEMO1* | 0.83 | −0.34 | −0.60 | −0.43 |
| NFATC3* | 0.75 | −0.58 | −0.8 | −0.59 |
| NFATC4 | −0.53 | 0.68 | 0.73 | 0.53 |
| SPTAN1 | −0.30 | 0.64 | 0.69 | 0.60 |
| SPTBN4 | −0.56 | 0.60 | 0.64 | 0.53 |
| SYNE1 | −0.51 | 0.58 | 0.66 | 0.52 |
| TIAM2 | −0.44 | 0.65 | 0.75 | 0.62 |
| TINKS | −0.53 | 0.43 | 0.62 | 0.53 |
| TUBB* | 0.71 | −0.29 | −0.63 | −0.38 |
| TRIO | −0.43 | 0.66 | 0.69 | 0.62 |
| XRN2* | 0.66 | −0.41 | −0.70 | −0.50 |
| YWHAE* | 0.62 | −0.36 | −0.35 | −0.62 |

Pearson's R, * PBMC microarray detection

### Discussion

Paired PBMC samples from patients hospitalized with acute psychosis were analyzed for potential treatment-response biomarkers of psychosis using gene chip analysis and RT-qPCR. Initial findings are presented demonstrating that decreases in DISC1 isoforms that include the region beyond exon 3 were associated with treatment in 11 out of 12 patients with primarily schizophrenia treated with a variety of antipsychotics. One paired sample of the 12 examined, from patient in Entry 3 of Table 1, had an increase in DISC1 transcripts during the course of treatment. Psychotic symptoms of patient in Entry 3 of Table 1 were substantially improved between paired sample collections. Without being bound by any theory, it is believed that this increase in DISC1 is a result of the patient taking benztropine, an inhibitor of dopamine uptake, during hospitalization. Whereas patient in Entry 1 of Table 1 whose second sample had decreased levels of DISC1 transcript as determined by gene chip analysis alone had benztropine listed as a discharge medication and it is not clear if the patient had started taking this medication when the paired samples were collected Likewise, patient in Entry 6 of Table 1 had amantadine, which may increase synthesis and release of dopamine, listed as a discharge medication. Patient in Entry 9 of Table 1 started levodopa, a dopamine precursor, the same day that she was enrolled and decreased levels of DISC1 were found between her paired samples. Immune response and genetics might influence the relationship between DISC1 levels in PBMCs and treatment of psychosis as well.

Without being bound by any theory, it is believed that the mechanism by which levels of DISC1 in PBMCs is associated with antipsychotic treatment involves the DRD2/GSK3β pathway. The antipsychotics that the patients were administered during hospitalization worked as DRD2 antagonists in the brain. Antipsychotics bind to DRD2 receptors on PBMCs if they were expressed. Detection of lymphocyte DRD2 is controversial and DRD2 was not found using immunocytochemisty whereas D3 and D4 were readily detected. However, lymphocyte DRD2 transcripts have been detected and analysis of data from CD34+ cells isolated from bone marrow confirmed that transcripts of 27 proteins implicated in the DRD2/DISC1 pathway were present. Significant correlations with DRD2 and DISC1-biomarker were identified with 8 of the 27 genes examined in the DRD2 pathway. Lymphocytes expressing pathways found in the brain can act as a surrogate of the brain through exposure as part of regular immune surveillance of the brain. In this context one can expect lymphocytes entering the brain of patients experiencing acute psychosis to have a greater response to dopamine in the brain because their DRD2 receptors are inadequately blocked. As a result, transcript levels and activity of key regulatory proteins downstream of DRD2 would be expected to be aberrant. After the patient receives the correct dose of antipsychotic medication, transcript levels and activity of the key regulatory proteins become normal because the pathway no longer is over stimulated by dopamine in the brain. Activity of GSK3β has been implicated as part of the DRD2 pathway and the N terminal encoded by exon 2 of the full-length DISC1 has been shown to directly bind GSK3β and inhibit its activity. The differentially expressed transcripts of DISC1 identified herein include exon 2 and if translated is able to bind and inhibit GSK3β. Five of the 12 patients were treated with either lithium or valproic acid that have been implicated in GSK3β regulation as well.

As the results show, DISC1 isoform levels in PBMCs can be used as a surrogate for what is happening in the brain in patients with schizophrenia being treated with antipsychotic medication. It is believed that these medications exert their effect through the DRD2 pathway and GSK3β. The treatment-response biomarker assay detects isoforms of DISC1 that include the 3′UT region of exon 3.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. (canceled)

2. A method for determining effectiveness of a pharmacotherapy in a schizophrenic patient comprising:
a) determining the expression level of a biomarker associated with a pharmacotherapy treatment response in schizophrenia patients from a biological sample obtained from the patient after initiation of the pharmacotherapy, wherein the biomarker comprises at least one DISC1 isoform; and
b) comparing the expression level of the biomarker detected in the biological sample to a level of expression of the biomarker in a control to determine the patient's response to the pharmacotherapy treatment, wherein the level of expression of the biomarker in the control comprises the level of expression of the biomarker in the patient during an acute psychosis,
and wherein a lower level of expression of the biomarker in the biological sample compared to the level of expression of the biomarker in the control is indicative of the effectiveness of pharmacotherapy.

3. A method for determining responsiveness of a subject suffering from a mental or neurological disorder to a pharmacotherapy treatment of a DRD2 antagonist, said method comprising:
a) determining the expression level of a biomarker associated with a pharmacotherapy treatment response in schizophrenia patients from a biological sample obtained from the patient, wherein the biomarker comprises at least one DISC1 isoform; and
b) comparing the expression level of the biomarker detected in the biological sample to a level of expression of the biomarker in a control to determine the patient's response to the pharmacotherapy treatment.

4. The method of claim 3, wherein the mental or neurological disorder comprises schizophrenia, bipolar disorder, or major depressive disorder.

5. The method of claim 3, wherein said step of determining the expression level of the biomarker comprises analyzing a plurality of DISC1 isoforms.

6. The method of claim 3, wherein said step of determining the expression level of the biomarker comprises analyzing at least five DISC1 isoforms.

7. The method of claim 3, wherein the biomarker comprises DISC1 variant q, AK025293, AK023443, TSNAX-DISC1, TSNAX-DISC1 variant kaje, or a combination thereof.

8. The method of any of claim 3, wherein said step of determining the expression level of the biomarker comprises determining the level of mRNA, protein, or gene expression associated with the DISC1 isoform.

9. The method of claim 3, wherein said step of determining the expression level of the biomarker comprises determining the level of mRNA associated with the DISC1 isoform.

10. The method of claim 3, wherein the biomarker comprises at least one DISC1 isoform comprising a variant in exon 3 of DISC1 gene.

11. The method of claim 3, wherein the biological sample comprises peripheral blood mononuclear cells of the patient.

12. The method of claim 3, wherein the biological sample comprises a peripheral blood lymphocyte of the patient.

13. The method of claim 3, wherein the level of expression of the biomarker in the control comprises the level of expression of the biomarker in non-schizophrenic subjects.

14. The method of claim 3, wherein the level of expression of the biomarker in the control comprises the level of expression of the biomarker in the patient during an acute psychosis, wherein a lower level of expression of the biomarker in the biological sample compared to the level of expression of the biomarker during acute psychosis is indicative of the patient's positive response to the pharmacotherapy.

15. A microarray comprising a plurality of oligonucleotides that are capable of detecting expression level of at least two DISC1 isoforms selected from the group consisting of DISC1 variant q, AK025293, AK023443, TSNAX-DISC1, and TSNAX-DISC1 variant kaje.

16. The microarray of claim 15, wherein said microarray is capable of detecting the expression of at least three DISC1 isoforms.

17. The microarray of claim 15, wherein said microarray is capable of detecting the expression of all five DISC1 isoforms.


SEQUENCE LISTING (24 sequences)

SEQ ID NO: 1 - DNA, 22 bp, Artificial Sequence
  Forward Primer I Sequence
ttgggacaccctgctcaggaat

SEQ ID NO: 2 - DNA, 24 bp, Artificial Sequence
  Reverse Primer I Sequence
gtattcttccacgtgggtcctctt

SEQ ID NO: 3 - DNA, 22 bp, Artificial Sequence
  One of two forward primer set II sequence
cagcccaggcggagcgggagga

SEQ ID NO: 4 - DNA, 22 bp, Artificial Sequence
  Second Forward Primer II Sequence
ggagctggcagcggggcgcatg

SEQ ID NO: 5 - DNA, 26 bp, Artificial Sequence
  One of Reverse Primer set II Sequence
ccctggtagagatgatcagaggaaca

SEQ ID NO: 6 - DNA, 22 bp, artificial
  Second Reverse Primer set II Sequence
gggacatgatgacaaaacaatc

SEQ ID NO: 7 - DNA, 26 bp, Artificial Sequence
  Forward Primer Set III sequence
tcacccaagatgcctccagaagaann

SEQ ID NO: 8 - DNA, 26 bp, Artificial Sequence
  One of Reverser Primer Set III Sequence
ccctggtagagatgatcagaggaaca

SEQ ID NO: 9 - DNA, 22 bp, Artificial Sequence
  Alternative Reverse Primer III Sequence
gggacatgatgacaaaacaatc

SEQ ID NO: 10 - DNA, 26 bp, Artificial Sequence
  Forward Primer set IV Sequence
tcacccaagatgcctccagaagaann

SEQ ID NO: 11 - DNA, 26 bp, Artificial Sequence
  Reverse Primer set IV Sequence
tcacccaagatgcctccagaagaann

SEQ ID NO: 12 - DNA, 23 bp, Artificial Sequence
  Forward Primer Sequence for Set V
gaagaagttcagttttgaaacag

SEQ ID NO: 13 - DNA, 21 bp, Artificial
  Reverse Primer Sequence for set V
aactattccctggtagagatg

SEQ ID NO: 14 - DNA, 24 bp, Artificial Sequence
  One Forward Primer Sequence for Set VI
tttcccaggttccctcggcctgta

SEQ ID NO: 15 - DNA, 24 bp, Artificial Sequence
  Another Forward Primer Sequence for set VI
tgaggaacatggacgagatgggaa

SEQ ID NO: 16 - DNA, 26 bp, Artificial Sequence
  One Reverse Primer Sequence for Set VI
ccctggtagagatgatcagaggaaca

SEQ ID NO: 17 - DNA, 24 bp, Artificial Sequence
  Another Reverse Primer Sequence for Set VI
tccctggtagagatgatcagagga

SEQ ID NO: 18 - DNA, 29 bp, Artificial Sequence
  Forward Primer Sequence of Set VII
tcacagtgcctttacctcaagctttagct

SEQ ID NO: 19 - DNA, 20 bp, Artificial Sequence
  Reverse Primer Sequence of Set VII
atgggcagacaggtggcaag

SEQ ID NO: 20 - DNA, 20 bp, Artificial Sequence
  Forward Primer Sequence of Set VIII
ggcagatggaggtaatatcc

SEQ ID NO: 21 - DNA, 21 bp, Artificial Sequence
  Reverse Primer Sequence of Set VIII
aactattccctggtagaggtg

SEQ ID NO: 22 - DNA, 20 bp, Artificial Sequence
  Forward Primer Sequence of Set IX
cggctaccacatccaaggaa

SEQ ID NO: 23 - DNA, 18 bp, Artificial Sequence
  Reverse Primer Sequence of Set IX
gctggaattaccgcggct

SEQ ID NO: 24 - DNA, 1270 bp, Homo sapiens
acgtgagaggagacttccggccactgcgttgtagtcggcccggctgcaaagcgtttttctgcaggctgttttcccaggttccctcggcctgtacctcgcgcactcctcttgctccaggtccttcagtctccgctcgtctcaccgtaggctgtgacgacatgagcaacaaagaaggatcaggagggttcaggaaaaggaagcatgacaatttcccacataaccaaagaagagaagggaaggatgttaattcatcttcacccgtgatgttggcctttaaatcatttcagcaggaacttgatgcaaggcatgacaaatatgagagacttgtgaaacttagtcgggatataactgttgaaagtaaaaggacaatttttctcctccataggattacaagtgctcctgatatggaagatatattgactgaatcagaaattaaattggatggtgtcagacaaaagatattccaggtagcccaagagctatcaggggaagatatgcatcagttccatcgagccattactacagaagtttacaggcaagatcctactcacagaggctcttgggatcaaggctgacagaagctccatctgaagacttttatactatagaggcagaaaaagagtgctgcgggactgcctgctgagaaaccggaggcagatggaggtaatatccttaagattaaaacttcagaaacttcaggaagatgcagttgagaatgatgattatgataaaggtgagttttaatttgtttattgattgttttgtcatcatgtcccaattttctttccatctttactcatatctaccttttgaatcccaaaagaattgtacaatctgttcctctgatcatctctaccagggaatagttgactcttttacagcattattgttttgtagatttcaaagtacttcatgaacattaatcctttgggttataaatataaccttatcaattgtccagaaacacttagcatactcacacaataaaaattatattagcttgccacctgtctgcccatggtgtgatgtatctataatccacttgttcataaaaaatattcgtctgacacctatgatatgctagggaatatgggagacaataggaaagtaagacagacatggtatctgcccttgtgatgccagtaaacttaagccttcatggggctctctgacttcataatttccagaaccagagataggaaatgaggtgaatttgagaaatgtcagactgtgccaaagggggtcacatgcatcaaatttccacatacat